(19) United States
(12) Patent Application Publication
(10) Pub. No.: US 2015/0323511 A1
(43) Pub. Date: Nov. 12, 2015

(54) SENSOR CALIBRATION METHOD AND APPARATUS

(71) Applicant: Sphere Medical Limited, Cambridge Cambridgeshire (GB)

(72) Inventors: Stuart HENDRY, Cambridgeshire (GB); Gavin TROUGHTON, Cambridgeshire (GB)

(21) Appl. No.: 14/411,423

(22) PCT Filed: Jul. 3, 2013
(86) PCT No.: PCT/GB2013/051766

§ 371 Date: Dec. 26, 2014

(30) Foreign Application Priority Data
Jul. 4, 2012 (GB) 1211881.6

(51) Int. Cl. G01N 33/00; G01N 33/48

(52) U.S. Cl. CPC G01N 33/0006; G01N 33/48

(57) ABSTRACT

Disclosed is a method of calibrating an apparatus comprising at least one sensor for detecting one or more analytes of interest in a sample, the method comprising measuring a first set of responses of the at least one sensor to at least one first calibration solution having a known composition of the one or more analytes of interest; measuring a second response of the at least one sensor to a second calibration solution having an approximately known composition of the one or more analytes of interest; determining the composition of the second calibration solution from the difference between the first set of responses and the second response; and periodically calibrating the at least one sensor with the second calibration solution using said determined composition. An apparatus and computer program product for executing this method are also disclosed.

### FIELD OF THE INVENTION

The present invention relates to a method of calibrating an apparatus comprising at least one sensor for detecting one or more analytes of interest in a sample.

The present invention further relates to an apparatus comprising a processor, a memory operatively coupled to the processor and at least one sensor for detecting one or more analytes of interest in a sample operatively coupled to the processor.

### BACKGROUND OF THE INVENTION

In order to obtain an accurate reading of the presence of one or more analytes of interest in a sample using an apparatus comprising one or more sensors for detecting one of the analytes of interest, it is necessary to (periodically) calibrate the one or more sensors with one or more calibration solutions having known concentrations of the one or more analytes of interest. The response of the one or more sensors to the calibration solution(s) is measured and calibration coefficients for each sensor are derived from the sensor response and the known concentration of the corresponding analyte of interest. The thus obtained calibration coefficients are subsequently used to determine the quantity of an unknown amount of the one or more analytes of interest in a sample from the sensor response to that sample.

Although the concept of calibration is straightforward enough, its practical implementation is not without problems. The concentrations of the one or more analytes of interest in the calibration solution(s) must be accurately known, as the accuracy of the subsequent measurements of samples to which the apparatus is exposed depends on the accuracy of the concentrations to which the calibration coefficients will be correlated. This is particularly important in medical application domains, where the apparatus may for instance be used to monitor analyte levels such as potassium, glucose, pH, oxygen (O₂), carbon dioxide (CO₂) and so on in a bodily fluid of the patient such as the patient's blood.

To this end, calibration solutions are typically manufactured in a batch, after which they are packaged into individual units and shipped to the end user. During transit or storage, the packaged calibration solutions are typically exposed to varying environmental conditions such as temperature and pressure, which can affect the concentrations of analytes of interest in the calibration solution. This is particularly problematic for calibration solutions into which gases such as O₂ and/or CO₂ are dissolved because the concentration of a dissolved gas is highly sensitive to such environmental variations. Such problems can for instance occur if the calibration solution is packaged in a gas-permeable container or in a container with a headspace, which allows for the gas concentrations in the calibration solution to change freely upon such changes in the environmental conditions.

In order to avoid such problems, calibration solutions may be packaged in containers that do not allow such variations when the container is exposed to the aforementioned variations in environmental conditions. Glass vials filled without head space are particularly suitable for such purposes as they maintain the calibration solution integrity due to their rigidity and negligible diffusion rates through the glass.

However, such containers are not ideal from the perspective of the end user especially if they have to be used frequently; they are cumbersome to open with the risk of glass particles generated during the breaking of the glass vial finding their way into the apparatus, which is entirely unacceptable if the apparatus is connected to a patient. Also, such packaging can be quite costly compared to less robust packaging materials such as polymer-based syringes, which is also undesirable to the end user from an economic perspective.

### SUMMARY OF THE INVENTION

The present invention seeks to provide a method of calibrating an apparatus comprising at least one sensor for detecting one or more analytes of interest in a sample in which the need to use cumbersome calibration solution packaging can be reduced.

The present invention further seeks to provide an apparatus comprising at least one sensor for detecting one or more analytes of interest in a sample that periodically can be accurately calibrated without the need to use cumbersome calibration solution packaging for each calibration step.

The present invention further seeks to provide a computer program product that allows for the method of the present invention to be executed on the apparatus of the present invention.

In accordance with an aspect of the present invention, there is provided a method of calibrating an apparatus comprising at least one sensor for detecting one or more analytes of interest in a sample, the method comprising measuring a first set of responses of the at least one sensor to at least one calibration solution having a known composition of the one or more analytes of interest; measuring a second response of the at least one sensor to a second calibration solution having an approximately known composition of the one or more analytes of interest; determining the composition of the second calibration solution from the difference between the first set of responses and the second response; and periodically calibrating the at least one sensor with the second calibration solution using said determined composition. The first set of responses may comprise a single response in case of a single calibration solution with a known composition being used, or may comprise a plurality of responses in case of a multi-point calibration in which a plurality of calibration solutions with different known composition are used, such as two responses in case of a two-point calibration using two different calibration solutions each having a known composition. In an embodiment, the number of responses in the set of responses equals the number of different calibration solutions with known compositions.

The present invention is based on the insight that a combination of a first set of calibration solutions each having a well-defined analyte composition, e.g. a first calibration solution stored in a glass vial or the like, can be used to determine the exact composition of a second calibration solution that is stored in packaging susceptible to undergoing compositional changes due to variations in environmental conditions to which the packaging has been exposed. In order words, although such a second calibration solution is likely to have an approximately known analyte composition only its composition can nevertheless be accurately determined by comparing the respective sensor responses to the first set of calibration solutions, i.e. one or more calibration solutions and the second calibration solution.

As the end user typically stores the second calibration solution, e.g. separate packages of the second calibration solution belonging to the same manufacturing batch, under sufficiently constant environmental conditions, e.g. substantially constant temperature and pressure, it may therefore be assumed that the composition of the second calibration solution will not change during the period of use of the apparatus, such that the determined composition of the second calibration solution remains valid during the period of use of the apparatus, thereby guaranteeing accurate periodic (re)calibration of the one or more sensors of the apparatus despite the use of a calibration solution in a container that allows changes in the analyte composition in response to environmental changes, thus reducing the need to use cumbersome calibration solution packages.

In an embodiment, the step of measuring a first set of responses of the at least one sensor to at least one calibration solution having a known composition of the one or more analytes of interest further comprises calibrating the at least one sensor with said at least one calibration solution. This further improves the accuracy of the calibration method.

This embodiment may further comprise periodically calibrating the at least one sensor with the at least one calibration solution, wherein the calibration frequency using the second calibration solution is higher than the calibration frequency using the at least one calibration solution. This yet further improves the accuracy of the calibration method.

In a further embodiment, the method further comprises repeating the steps of rejecting the second calibration solution if the difference between at least one response from the first set of responses and the second response exceeds a defined threshold; and measuring a second response of the at least one sensor to another volume of the second calibration solution until said difference falls within said defined threshold.

This embodiment is based on the insight that the composition of the second calibration solution, which had a well-defined analyte composition at the point of manufacture, can only vary within certain limits, for instance because the expected variations in environmental conditions are limited. Therefore, if the determined actual analyte composition of the second calibration solution falls outside the compositional range that can be reasonably expected, this is an indication that the second calibration solution has been subjected to extreme environmental conditions or that an error has occurred during manufacture. Either way, as the second calibration solution can no longer be trusted, it may be rejected and replaced by another instance of the second calibration solution, e.g. a different package from the same or a different manufacturing batch.

The step of periodically calibrating the at least one sensor with the second calibration solution using said determined composition may further comprise predicting a response of the at least one sensor to the second calibration composition; comparing the predicted response to the actual response of the at least one sensor to the second calibration solution; and rejecting the calibration step if the difference between the predicted response and the actual response exceeds a defined further threshold. For instance, the step of predicting a response of the at least one sensor to the second calibration composition may comprise predicting said response using a sensor drift model. This has the advantage that unusual sensor behavior or unusual discrepancies in the expected composition of the second calibration solution can be detected, thus further improving the accuracy of the calibration method.

The method of the present invention is particularly suitable for calibration solutions in which one or more analytes of interest comprise a gas, such as CO₂ or O₂ as such calibration solutions are particularly sensitive to changes in environmental conditions, although the present invention is not limited to gas-containing calibration solutions.

The method of the present invention allows the use of a second calibration solution stored in a gas-permeable container, as the variations in the composition of this calibration solution are compensated for by the method of the present invention.

The method of the present invention is particularly suited to an apparatus adapted to analyze a bodily fluid sample, where it may be particularly important to provide a user-friendly way of calibrating the apparatus whilst at the same time reducing the risk that a patient is exposed to debris from an opened calibration solution package.

In accordance with another aspect of the present invention, there is provided an apparatus comprising a processor, a memory operatively coupled to the processor and at least one sensor for detecting one or more analytes of interest in a sample operatively coupled to the processor, wherein the processor is adapted to measure a first set of responses of the at least one sensor to at least one calibration solution having a known composition of the one or more analytes of interest; measure a second response of the at least one sensor to a second calibration solution having an approximately known composition of the one or more analytes of interest; determine the composition of the second calibration solution from the difference between the first set of responses and the second response; and periodically calibrate the at least one sensor upon exposure of the at least one sensor to the second calibration solution using said determined composition.

As already explained in more detail above, such an apparatus is advantageous as it can be accurately calibrated using calibration solutions of which the exact composition cannot be guaranteed to a sufficient degree of certainty.

The processor may be adapted to store the determined concentration in said memory and to retrieve said determined composition from said memory during said periodic calibration such that recalibration of the one or more sensors may be performed in an automated fashion.

In an embodiment, the processor is further adapted to predict a response of the at least one sensor to the second calibration composition; compare the predicted response to the actual response of the at least one sensor to the second calibration solution; and reject the second calibration solution if the difference between the predicted response and the actual response exceeds a defined further threshold. As previously explained, this reduces the risk of inaccurate calibration due to unreliable calibration solutions.

Preferably, the apparatus is adapted to analyze a body fluid sample, wherein at least one of the analytes of interest comprises a gas.

In accordance with yet another aspect of the present invention, there is provided a computer program product comprising a computer-readable medium comprising computer program code for, when executed on the processor of the apparatus of the present invention, causing its processor to execute the steps of the method of the present invention. This amongst others has the advantage that the calibration method of the present invention may be retrofitted onto an existing apparatus.

### BRIEF DESCRIPTION OF THE EMBODIMENTS

Embodiments of the invention are described in more detail and by way of non-limiting examples with reference to the accompanying drawings, wherein:

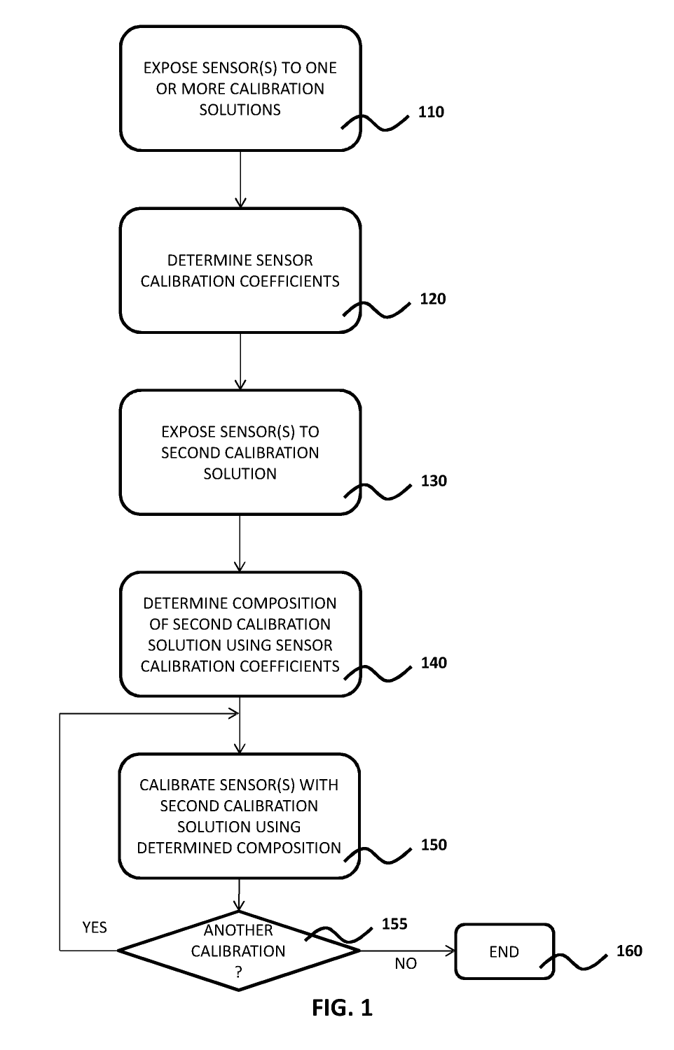

FIG. 1 depicts a flow chart of an example embodiment of the method of the present invention;

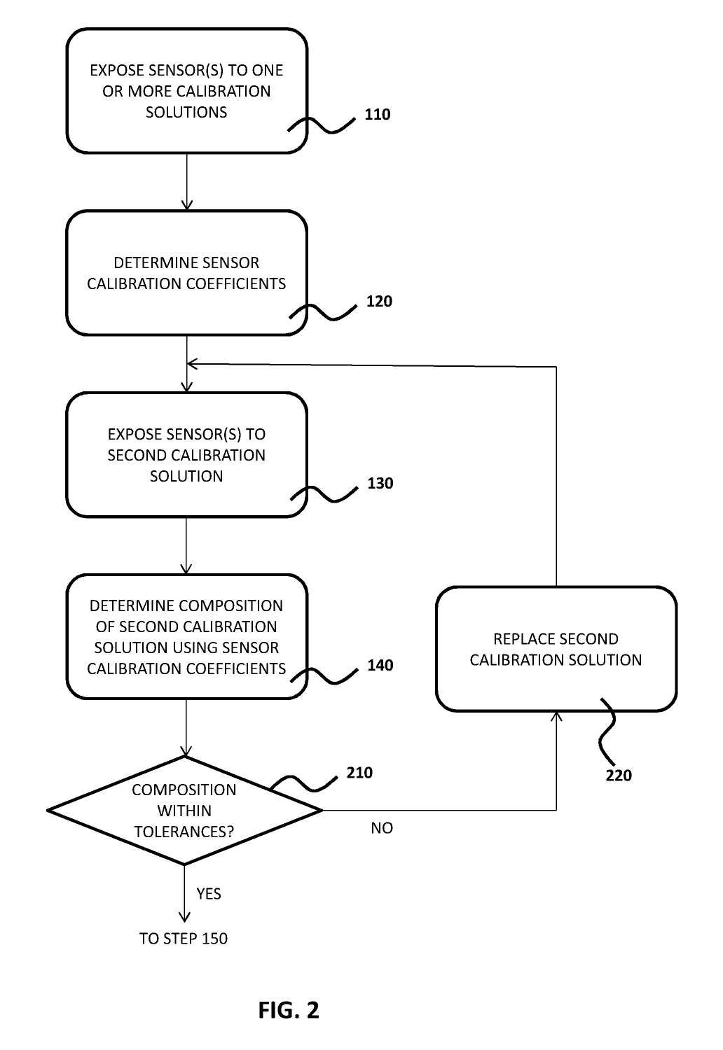

FIG. 2 depicts a flow chart of an aspect of another example embodiment of the method of the present invention;

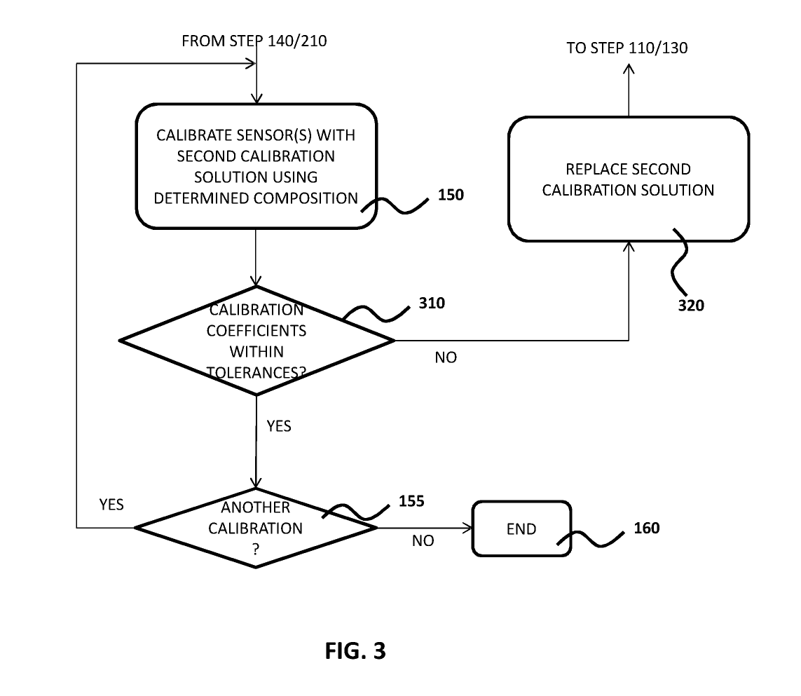

FIG. 3 depicts a flow chart of an aspect of yet another example embodiment of the method of the present invention; and

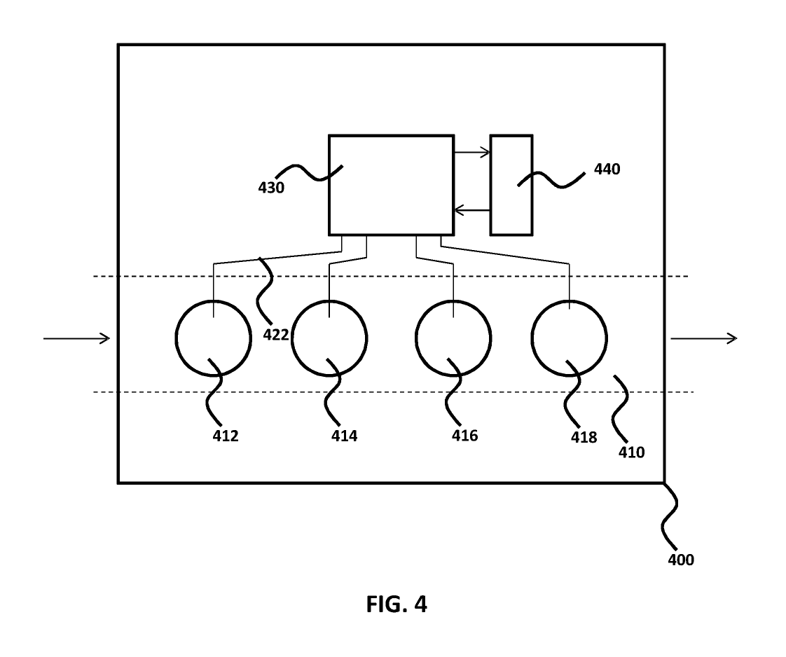

FIG. 4 schematically depicts an example embodiment of the apparatus of the present invention.

### DETAILED DESCRIPTION OF THE DRAWINGS

It should be understood that the Figures are merely schematic and are not drawn to scale. It should also be understood that the same reference numerals are used throughout the Figures to indicate the same or similar parts.

FIG. 1 depicts a flow chart of a non-limiting example embodiment of the method of the present invention, which will be explained with the aid of FIG. 4, in which an example embodiment of an apparatus 400 of the present invention is shown. The apparatus 400 comprises a sample chamber 410 comprising at least a first sensor 412 for detecting a first analyte of interest in a sample, at may optionally comprise further sensors for detecting further analytes of interest in the same or a different sample. By way of non-limiting example, the apparatus 400 comprises a second sensor 414 for detecting a second analyte of interest, a third sensor 416 for detecting a third analyte of interest and a fourth sensor 418 for detecting a second analyte of interest, although it should be appreciated that the sample chamber 410 may comprise any suitable number of sensors.

The sample chamber 410 may be adapted to evaluate a stationary sample, in which case it may comprise an inlet. Alternatively, the sample chamber 410 may be adapted to evaluate a flowing sample, in which case the sample chamber 410 may comprise an inlet and an outlet as indicated by the arrows on either side of the sample chamber 410, e.g. a flow cell or flow channel. The sensor 412 and optional sensors 414, 416 and 418 may be of any suitable design. It will be appreciated that the design of the sensor(s) and the sample chamber 410 are outside the scope of the present invention. As such designs are well-known per se, they will not be further discussed at this stage for the sake of brevity only.

The apparatus 400 further comprises a processor 430 that is conductively coupled to the first sensor 412 and, if present, further sensors 414, 416 and 418 via respective conductors 422, e.g. metal tracks of an integrated circuit or printed circuit board, or via a cable comprising conductive wires 422 in case the processor 430 is physically separated from the sample chamber 410, e.g. is not integrated on the substrate or carrier as the sample chamber 410. One or more memories 440 may be present in the apparatus 400, which may be operatively coupled to the processor 430 such that the processor 430 can read from the memory 440 and write to the memory 440 when necessary. The processor 430 is typically programmed to calculate calibration coefficients during a calibration step and to calculate concentrations of an analyte of interest from a measured sensor response and the calibration coefficients during a measurement step as is well known per se to the person skilled in the art.

At this point, it is noted that although a single processor 430 is shown in FIG. 4, the processor 430 may have a distributed architecture, e.g. may comprise a first portion for processing the analog or digital signals from the one or more sensors of the apparatus 430, e.g. by converting the analog signals into digital signals, and a digital signal processing portion for interpreting the sensor signals. Similarly, the memory 440 may have a distributed architecture, and at least part of the memory 440 may reside on the processor 430, e.g. in the form of a cache. In an embodiment, the memory 440 may comprise a read-only portion and a further portion into which data can be written as well as read from, e.g. a ROM portion and a RAM or flash memory portion. As such architectures are known per se, many variations will be immediately apparent to the skilled person.

In an embodiment, the apparatus 400 is a system for monitoring analyte concentrations in a bodily fluid such as saliva, blood or urine. In a specific embodiment, the apparatus 400 is an in-line blood monitor system, in which at least the sample chamber 410 and the first sensor 412, and, if present, one or more of the optional sensors 414, 416 and 418 are placed in a line connected to the vein or artery of a patient. In this embodiment, the sensors may be adapted to monitor analytes of interest in the blood of the patient, e.g. Na⁺, K⁺, glucose, CO₂, O₂ and hematocrit levels, for instance. However, it should be understood that the present invention is not limited to an apparatus 400 for medical application domains.

Now, upon returning to FIG. 1, the method commences in step 110 by exposing the one or more sensors present in the sample chamber 410 to at least one calibration solution comprising well-defined concentrations of the one or more analytes of interest. This for instance can be guaranteed by providing the calibration solution in a container that can be exposed to changes in the environmental conditions to which it is exposed, e.g. during travel or storage, without it affecting the concentrations of the analyte(s) of interest in the calibration solution stored therein. This is particularly relevant if the analyte(s) of interest contain one or more gases, such as CO₂ and O₂. A non-limiting example of such a container is a glass vial, although other suitable gas-impermeable containers or canisters will be immediately apparent to the skilled person.

In an embodiment, step 110 comprises exposing the one or more sensors present in the sample chamber 410 to at least two different calibration solutions each comprising well-defined concentrations of the one or more analytes of interest. In this embodiment, the method implements a multi-point, e.g. a two-point, calibration of the one or more sensors.

In a next step 120, the processor 430 measures the response of the sensor(s) to the analyte(s) of interest and determines the calibration coefficients from these responses. The processor 430 may be triggered to do so by program code stored in the memory 440. In order to calculate the calibration coefficients, the processor 430 may receive the concentrations of the analytes of interest in the first calibration solution in any suitable manner, e.g. through user input via a user interface (not shown), via a near-field communication or other radio-frequency communication with a chip containing this information in or on the packaging of the first calibration solution and so on. As the determination of such calibration coefficients is well-known per se, this will not be explained in further detail for the sake of brevity only.

The processor 430 may store the calibration coefficients and/or the analyte concentration information for the first calibration solution in the memory 440.

If desired, the sample chamber 410 may be flushed at this stage to remove the (first) calibration solution(s) from the sample chamber 410.

In the next step 130, the one or more sensors in the sample chamber 410 are exposed to a second calibration solution that may have undergone changes in its analyte composition, e.g. because it is stored in a gas-permeable container that has been subjected to non-trivial changes in the environmental conditions to which it has been exposed, e.g. temperature and/or pressure changes during storage or transport.

Typically, several instances of the second calibration solution are purchased together, e.g. by purchasing a plurality of containers each comprising a volume of the same calibration solution batch, or a single container comprising multiple volumes of the calibration solution, such that each of said volumes has been exposed to the same changes in the environmental conditions, such that it can be assumed with a high level of confidence that if the actual compositions of the various volumes of the second calibration solution have deviated from their original composition, these actual compositions all differ in the same manner from this original composition. Moreover, as the plurality of containers comprising the second calibration solution are typically stored by the end user under the same well-controlled environmental conditions, e.g. the same substantially constant temperature and pressure, it can furthermore be assumed with a high level of confidence that the compositions of the various volumes of the second calibration solution are constant over the duration of the use of the apparatus 400.

Alternatively, the multiple volumes of the second calibration solution may be stored in the same container, e.g. a fluid bag, in which case subsequent volumes of the second calibration fluid may be drawn from the same container. In this case, at least some embodiments of the method of the present invention may be used to detect environmentally induced variations in the composition of the second calibration solution within a single container.

In the next step 140, the actual composition of a volume of the second calibration solution is determined by the processor 430 calculating the concentrations of the one or more analytes of interest in the second calibration solution from the one or more sensor responses to the second calibration solution and the calibration coefficients determined in step 120. The calculated concentrations of the analytes are subsequently stored in the memory 440 by the processor 430. The apparatus 400 is now ready to be periodically recalibrated with a volume of the second calibration solution from the same batch as used in step 130.

To this end, the method proceeds to step 150, e.g. after a predefined period of time during which the apparatus 400 may have been exposed to one or more samples, in which the one or more sensors in the sample chamber 410 are exposed to a further volume of the second calibration solution from the same batch as the volume of the second calibration solution used in step 130. The processor 430 collects the sensor responses of the apparatus 400 to the one or more analytes of interest in the second calibration solution, retrieves the previously calculated concentrations of the analytes of interest from memory 440 and calculates the updated calibration coefficients from the sensor responses and the previously calculated concentrations. The processor 430 may subsequently store the updated calibration coefficients in the memory 440 for use during subsequent measurement performed with the apparatus 400 during exposure to one or more samples.

It is subsequently checked in step 155 if the calibration step 150 needs to be repeated, e.g. because a certain amount of time has elapsed during the use of the apparatus 400, or because of any other suitable determination criteria. If this is the case, the method reverts back to step 150 for another recalibration of the sensor with a fresh volume of the second calibration solution from the same batch. Otherwise, the method may terminate in step 160, e.g. because the use of the apparatus 400 is terminated.

Although not explicitly shown in FIG. 1, from time to time the one or more sensors of the apparatus 400 may also be recalibrated using (a) fresh volume(s) of the calibration solution(s) having the known analyte concentrations, at which stage it may be decided to repeat steps 110-140 in order to further increase the confidence in the accuracy of the calibration method, e.g. to compensate for changes in the composition in the various volumes of the second calibration solution caused by an inadvertent change in the environmental conditions under which the second calibration solutions are stored.

In an embodiment of the present invention, the composition of the second calibration solution is correlated to the composition of the at least one calibration solution with known analyte concentrations, for instance by choosing the initial analyte concentrations in the second calibration solution such that they are less than a certain percentage different to the analyte concentrations in the first calibration solution. For instance, the initial analyte concentrations in the second calibration solution may be chosen to be the same as the analyte concentrations in one of the one or more well-defined calibration solutions.

One reason to choose the initial analyte concentrations of the second calibration solution to be similar if not the same to the analyte concentrations in the well-defined calibration solution is that the response of the sensor in the apparatus 400 for detecting the analyte of interest in the second calibration solution will be similar to the response of the sensor exposed to the same analyte in the well-defined calibration solution. As small deviations from the analyte concentration at which the sensor has been calibrated are likely to fall within the linear response regime of the sensor, the approximate concentration of the analyte of interest in the second calibration solution can be easily determined with a high degree of accuracy.

In contrast, large differences between the concentrations of an analyte of interest in the one or more calibration solutions having well-defined analyte concentrations on the one hand and second calibration solutions on the other hand may trigger a non-linear response from the sensor, e.g. because one of the concentrations falls outside the operating range of the sensor, which increases the risk of errors in the determination of the approximate concentration of the analyte of interest in the second calibration solution.

In addition, by choosing the initial analyte concentrations of the second calibration solution to be similar if not the same to the analyte concentrations in one of the one or more calibration solutions with well-defined analyte concentrations, the method shown in FIG. 1 may be adapted as shown in FIG. 2 to provide an additional check on the suitability of the second calibration solution, for instance to detect exposure of the second calibration solution to unacceptably large variations in the environmental conditions to which the second calibration solution has been exposed, and which may have permanently altered the composition of the second calibration solution, or to detect errors in the manufacturing process of the second calibration solution. To this end, the method is extended with an additional step 210, in which it is checked if the determined composition of the second calibration solution is similar enough to the composition of the well-defined calibration solution.

This may for instance be implemented by the processor 430 comparing the concentration of an analyte of interest as determined in step 140 with the known concentration of the analyte of interest of the one calibration solution of the one or more calibration solutions, and if the difference between these concentrations exceeds a defined threshold, the method may proceed to step 220 in which the volume of the second calibration solution is replaced with another volume of the second calibration from the same batch or a different batch in case the whole batch is rejected, after which the method returns to step 130. The threshold 130 may be defined in any suitable manner, e.g. by taking into consideration the likely and/or tolerable variations in the composition of the second calibration solution during transit and storage.

Alternatively, the processor 130 may record the values of the respective sensor responses to the one or more analytes of interest in the one or more calibration solutions and compare the recorded value(s) with the value of the sensor responses to the analyte(s) of interest in the second calibration solution in step 210 to determine if the second calibration solution has a composition that is similar enough to the composition of the first calibration solution to rule out any damage or manufacturing error. If it is decided that the second calibration solution is fit for purpose, the method may proceed to step 150 where it will continue as already explained in the detailed description of FIG. 1.

A similar test of the suitability of a volume of the second calibration solution may be applied at a later stage of the calibration method of the present invention, as is shown in FIG. 3. For instance, it is possible to predict an evolution in time of a sensor response to a known concentration of an analyte of interest, e.g. when (approximate) sensor drift characteristics are known, e.g. by applying a drift correction to the sensor signal measured in a previous calibration step 150 to obtain an expectation value of the calibration coefficients for the same concentration of analyte of interest in the second calibration solution during a subsequent calibration step 150.

To this end, the method may further comprise an additional step 310 in which the calibration coefficients calculated in the most recent step 150 are compared by the processor 430 to the expectation value of these calibration coefficients, and in case the difference between the calculated values and the expectation values exceeds a defined threshold, the most recently used second calibration solution is rejected in step 320 after which the method may return to step 130 to expose the apparatus 400 to another volume of the second calibration solution from the same batch. If this produces another rejection of the second calibration solution, this may be an indication that the sensor drift has deviated from expectation values, e.g. because of fouling of the sensor. In this scenario, it may be decided to subject the sample chamber 410 to a cleaning cycle after which the method returns to step 110.

Instead of comparing calibration coefficients in step 310, the processor 430 may alternatively compare the respective values of the sensor signals obtained in a previous and the most recent calibration step 140 to determine if the difference between these signals exceeds a defined threshold, as it is of course equally feasible to extrapolate a time-dependent change in the sensor response from the known sensor drift characteristics. Other possible variations will be immediately apparent to the skilled person.

It is not necessary that the sensor drift characteristics are known a priori. Alternatively, the processor 430 may extract the sensor drift characteristics from a trend in the sensor signals and/or calibration coefficients obtained in a series of calibration steps 140. In such a scenario, the additional steps 310 and 320 may not become available after a defined number of calibration steps 140 have taken place, said defined number being chosen such that the processor 430 can determine the sensor drift characteristics with sufficient accuracy.

Embodiments of the method of the present invention may take the form of computer program code for execution by the processor 430. Such program code may be stored on a computer-readable storage medium such as a CD, DVD, USB memory stick, MP3 player, memory, hard disk, network server and so on.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

1. A method of calibrating an apparatus (400) comprising at least one sensor (412, 414, 416, 418) for detecting one or more analytes of interest in a sample, the method comprising:
measuring (110, 120) a first set of responses of the at least one sensor to at least one calibration solution having a known composition of the one or more analytes of interest;
measuring (130) a second response of the at least one sensor to a second calibration solution having an approximately known composition of the one or more analytes of interest;
determining (140) the composition of the second calibration solution from the difference between the first set of responses and the second response; and
periodically calibrating (150) the at least one sensor with the second calibration solution using said determined composition.

2. The method of claim 1, wherein the at least one calibration solution comprises a pair of calibration solutions having different compositions of the one or more analytes of interest.

3. The method of claim 1, wherein the step of measuring (110, 120) the first set of responses of the at least one sensor to a first calibration solution having a known composition of the one or more analytes of interest further comprises calibrating the at least one sensor with said at least one calibration solution.

4. The method of claim 3, further comprising periodically calibrating the at least one sensor with the at least one calibration solution, wherein the calibration frequency using the second calibration solution is higher than the calibration frequency using the at least one calibration solution.

5. The method of claim 1, further comprising repeating the steps of:
rejecting (210) the second calibration solution if difference between at least one response from the first set of responses and the second response exceeds a defined threshold; and
measuring (130, 140) a second response of the at least one sensor to another volume of the second calibration solution;
until said difference falls within said defined threshold.

6. The method of claim 1, wherein the step of periodically calibrating (150) the at least one sensor with the second calibration solution using said determined composition comprises:
predicting a response of the at least one sensor to the second calibration composition;
comparing (310) the predicted response to the actual response of the at least one sensor to the second calibration solution; and
rejecting (320) the calibration step if the difference between the predicted response and the actual response exceeds a defined further threshold.

7. The method of claim 6, wherein the step of predicting a response of the at least one sensor to the second calibration composition comprises predicting said response using a sensor drift model.

8. The method of claim 1, wherein the one or more analytes of interest comprise a gas.

9. The method of claim 8, wherein the second calibration solution is stored in a gas-permeable container.

10. The method of claim 1, wherein the apparatus is adapted to analyze a bodily fluid sample.

11. An apparatus (400) comprising a processor (430), a memory (440) operatively coupled to the processor (410) and at least one sensor (412, 414, 416, 418) for detecting one or more analytes of interest in a sample operatively coupled to the processor, wherein the processor is adapted to:
measure (120) a first set of responses of the at least one sensor to at least one calibration solution having a known composition of the one or more analytes of interest; measure (130) a second response of the at least one sensor to a second calibration solution having an approximately known composition of the one or more analytes of interest;
determine (140) the composition of the second calibration solution from the difference between the first set of responses and the second response; and
periodically calibrate (150) the at least one sensor upon exposure of the at least one sensor to the second calibration solution using said determined composition.

12. The apparatus (400) of claim 11, wherein the processor (430) is adapted to store the determined concentration in said memory and (440) to retrieve said determined composition from said memory during said periodic calibration.

13. The apparatus (400) of claim 11 or 12, wherein the processor (430) is further adapted to:
predict a response of the at least one sensor to the second calibration composition;
compare (210, 310) the predicted response to the actual response of the at least one sensor to the second calibration solution; and
reject (210, 320) the second calibration solution if the difference between the predicted response and the actual response exceeds a defined further threshold.

14. The apparatus (400) of claim 11, wherein the apparatus is adapted to analyze of body fluid sample, and wherein at least one of the analytes of interest comprises a gas.

15. A computer program product comprising a computer-readable medium comprising computer program code for, when executed on a processor (430), causing the processor to execute the steps of
measuring (110, 120) a first set of responses of the at least one sensor to at least one calibration solution having a known composition of the one or more analytes of interest;
measuring (130) a second response of the at least one sensor to a second calibration solution having an approximately known composition of the one or more analytes of interest;
determining (140) the composition of the second calibration solution from the difference between the first set of responses and the second response; and
periodically calibrating (150) the at least one sensor with the second calibration solution using said determined composition.
